(19) United States
(12) Patent Application Publication
(10) Pub. No.: US 2026/0335092 A1
(43) Pub. Date: Oct. 29, 2026

(54) CASTING MOLD FOR FLASHLESS ACETABULAR SPACERS

(71) Applicant: Heraeus Medical GmbH, Wehrheim (DE)

(72) Inventors: Sebastian VOGT, Wehrheim (DE); Roman NASSUT, Wehrheim (DE); Thomas KLUGE, Wehrheim (DE)

(21) Appl. No.: 19/650,877

(22) Filed: Apr. 17, 2026

(30) Foreign Application Priority Data
Apr. 29, 2025 (EP) 25173108.9

(51) Int. Cl. A61F 2/30

(52) U.S. Cl. CPC A61F 2/30942; A61F 2002/30957; A61F 2310/00353

(57) ABSTRACT

A casting mold for producing an acetabular spacer, comprising a main body with a curved first mold half and a curved second mold half, a first extension and a second extension, a filling nozzle and a first passage, wherein the first mold half together with the second mold half defines a cavity, wherein the first extension and the second extension are arranged adjacent to each other on the main body and together define a cutting region of the main body, wherein the cutting region is arranged between the first extension and the second extension, and wherein the first passage is designed to allow excess bone cement to escape from the cavity of the casting mold.

### CROSS-REFERENCE TO RELATED APPLICATION

This application claims priority pursuant to 35 U.S.C. 119(a) to European Patent Application No. 25173108.9, filed Apr. 29, 2025, which application is incorporated herein by reference in its entirety.

### FIELD OF THE INVENTION

The present invention relates to the field of medical technology, in particular devices for producing medical implants, and the medical implants themselves that are producible therewith. Furthermore, the invention relates to a production method in which the devices according to the invention are used for producing medical implants.

### BACKGROUND

Spacers made of polymethyl methacrylate bone cement, which may contain one or more antibiotics, are successfully used in two-stage revision for periprosthetic joint infection. The spacers are used as temporary implants for the interim period until the revision joint prosthesis is implanted, following the removal of the infected joint prosthesis and subsequent debridement. During the interim period, one or more antibiotics can be released from the spacers and can help to mitigate the infection. The spacers are used for “deadspace management,” i.e., the treatment of surgically or infection-related cavities, and can prevent atrophy of the ligaments and muscles.

In two-stage revision for periprosthetic hip joint infection, the prosthesis stem with the prosthesis head and the hip socket are removed in a first operation. During the interim phase, hip joint spacers often consist of shafts made of polymethyl methacrylate (PMMA) and having a spherical or mushroom-shaped head. The heads have such a large diameter that they can be inserted directly into the acetabulum (hip socket) on the bone tissue. However, the problem is that the spacer heads can rub against the bone surface of the acetabulum during articulation.

Alternatively, so-called acetabular cup spacers were developed which replicate the hip socket. These are cup-shaped spacers with a concave surface to accommodate the spacer head and a convex back, which can be anchored in the acetabulum using polymethyl methacrylate bone cement dough.

European Patent No. 3,761,909 proposes an acetabular cup spacer that is hemispherical in shape. On the outside, recesses or extensions extend from the edge of the spacer, oriented toward the center of the hemisphere.

U.S. Patent No. 12,127,944 proposes a two-part casting mold consisting of a concave and a convex part. The concave part of the casting mold is connected to the convex part by screwing. The convex part contains an opening for injecting polymethyl methacrylate bone cement. Furthermore, tubular vents are arranged at the edge of the convex part. Flash can form on the cast spacer at the point where the two parts separate. Flash can be very sharp and can be removed mechanically only with considerable effort, especially if it has a thickness of approximately 0.3 mm or more. Such larger flash can either be broken off with pliers or trimmed off with milling cutters, which is tedious for the medical user and makes it difficult to achieve the desired shape of the spacer.

### SUMMARY

One object of the invention is to provide a casting mold which can be used in particular for the intraoperative production of acetabular cup spacers made of polymethyl methacrylate bone cement. The casting molds according to the invention are intended to avoid the formation of flash on the spacers as much as possible. Another object of the invention is to avoid air inclusions in the produced spacers. The invention also addresses the object of avoiding the formation of a sprue as much as possible, or at least keeping the sprue formed during molding as small and easily removable as possible.

Furthermore, an objective of the invention is to make the removal of the shaped spacer from the casting mold as easy, quick and safe as possible for the medical user. In particular, the risk of cuts, which can occur due to sharp-edged flash, is to be avoided. Another risk of cuts can arise if the medical user slips while cutting open a casting mold with a scalpel. In some embodiments, the casting mold according to the invention therefore allows safe and injury-free cutting of the casting mold. Another object of the invention is to produce the casting mold as cost-effectively as possible. This includes, for example, the selection of materials used for the casting mold and the simplest possible geometric design of the casting mold.

Individual objects, a plurality of objects or all of these objects are achieved by the casting molds described herein, in particular those described in the claims.

### PREFERRED EMBODIMENTS

In a first embodiment, one aspect of the invention relates to a casting mold for producing an acetabular spacer comprising a main body with a curved first mold half and a curved second mold half, a first extension and a second extension, a filling nozzle and a first passage, wherein the first mold half together with the second mold half define a cavity, wherein the first extension and the second extension are arranged adjacent to each other on the main body and together define a cutting region of the main body, wherein the cutting region is arranged between the first extension and the second extension, and wherein the first passage is designed to allow excess bone cement to escape from the cavity of the casting mold.

A second embodiment relates to a casting mold according to the first embodiment mentioned above, wherein the main body has a first wall thickness which is thicker than a second wall thickness of the cutting region.

A third embodiment relates to a casting mold according to the first or second embodiment mentioned above, wherein the first extension and the second extension are designed for guiding a scalpel along the cutting region to cut through the casting mold.

A fourth embodiment relates to a casting mold according to any of the preceding embodiments, wherein the first extension and/or the second extension comprises a tab to facilitate manually pulling apart the first mold half and the second mold half.

A fifth embodiment relates to a casting mold according to any of the preceding embodiments, wherein the first extension and/or the second extension extends at least 2mm, more preferably at least 3, 4, 5 or 6mm, outward from the main body.

A sixth embodiment relates to a casting mold according to any of the preceding embodiments, wherein the first extension and/or the second extension extends circumferentially around at least half of a circumference of the main body.

A seventh embodiment relates to a casting mold according to any of the preceding embodiments, further comprising a means for severing a sprue, wherein the means preferably comprises a movable element, the movable element further preferably being selected from a passage and a projection.

An eighth embodiment relates to a casting mold according to any of the preceding embodiments, further comprising a plug for closing the filling nozzle, wherein the first passage is preferably arranged in the plug.

A ninth embodiment relates to a casting mold according to the eighth embodiment, wherein the casting mold is designed and configured to sever a sprue by a rotational movement of the plug relative to the filling nozzle.

A tenth embodiment relates to a casting mold according to the eighth or ninth embodiment, wherein the plug comprises a first projection for severing a sprue, the first projection preferably being arranged at the first passage.

An eleventh embodiment relates to a casting mold according to embodiment ten, wherein the first projection comprises a beveled cutting edge.

A twelfth embodiment relates to a casting mold according to any of the preceding embodiments, wherein the filling nozzle comprises a second projection for severing a sprue, wherein the casting mold preferably further comprises a first projection according to the tenth embodiment, and wherein the casting mold is preferably designed and configured to sever a sprue by a rotational movement of the plug relative to the filling nozzle when the first projection is rotated relative to the second projection.

A thirteenth embodiment relates to a casting mold according to any of the preceding embodiments eight to twelve, wherein the first passage is arranged eccentrically in the plug.

A fourteenth embodiment relates to a casting mold according to any of the preceding embodiments eight to thirteen, wherein the filling nozzle further comprises a second passage, wherein the casting mold is designed and configured to arrange the first passage along a common axis with the second passage in a first rotational position of the plug, and to arrange the first passage outside a common axis with the second passage in a second rotational position of the plug.

A fifteenth embodiment relates to a casting mold according to any of the preceding embodiments, further comprising an operating element which is accessible from outside the casting mold and is designed and configured to sever a sprue.

A sixteenth embodiment relates to a casting mold according to any of the preceding embodiments, further comprising a vent opening, wherein the vent opening is preferably arranged between the first mold half and the second mold half.

A further aspect relates to the use of a casting mold according to any of the preceding embodiments for producing an acetabular spacer.

A further aspect relates to a method for producing an acetabular spacer, comprising the steps of:
providing a casting mold according to any of the embodiments 1 to 16;
filling the casting mold with bone cement;
hardening of the bone cement in the casting mold;
removing any sprue formed from the bone cement;
cutting open the main body of the casting mold in the cutting region (116);
separating the first mold half from the second mold half (112); and
removing the produced acetabular spacer from the cut-open casting mold.

### BRIEF DESCRIPTION OF THE DRAWINGS

FIG. 1 shows, by way of example, a first embodiment of the invention in cross section;

FIG. 2 shows, by way of example, an embodiment of a plug in cross section;

FIG. 3 shows, by way of example, a further embodiment of the invention in cross section;

FIG. 4 shows, by way of example, a further embodiment of the invention;

FIG. 5 shows, by way of example, a casting mold according to the invention which is filled with bone cement;

FIG. 6 shows, by way of example, a casting mold according to the invention, which has been cut open;

FIG. 7 shows a detail of a casting mold, which comprises a filling nozzle with an operating element, in a first position;

FIG. 8 shows a detail of a casting mold, which comprises a filling nozzle with an operating element, in a first position as a top view;

FIG. 9 shows a detail of a casting mold, which comprises a filling nozzle with an operating element, in a second position;

FIG. 10 shows a detail of a casting mold, which comprises a filling nozzle with an operating element, in a second position as a top view; and,

FIG. 11 shows a casting mold according to the invention and a bone cement dispensing device, which is arranged for connection to the casting mold.

### DETAILED DESCRIPTION OF THE INVENTION

With respect to the embodiments described herein, the elements of which "have", or "comprise", a particular feature (for example, a material), in principle, a further embodiment is always contemplated in which the relevant element consists solely of the feature, i.e., does not comprise any other constituents. The word "comprise" or "comprising" is used herein synonymously with the word "have" or "having".

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded on the basis of the context, it is possible in principle, and is hereby unambiguously contemplated, that features of different embodiments may also be present in the other embodiments described herein. Likewise, it is contemplated in principle that all features described herein in connection with a method are also applicable to the casting molds and spacers described herein, and vice versa. All these considered combinations are not explicitly listed in all cases, simply in order to keep the description brief.

A first aspect of the invention relates to a casting mold for the production of spacers. The casting mold according to the invention is particularly suitable for the production of an acetabular spacer. Such a spacer is also referred to herein as an acetabular cup spacer. Such a spacer replicates the shape of the hip socket of a hip joint. Acetabular spacers typically comprise a cup-shaped mold with a concave surface to accommodate a joint head or spacer head. Furthermore, an acetabular spacer typically comprises a convex back that can be attached to the acetabulum using bone cement.

The casting mold according to the invention comprises a main body formed by a curved first mold half and a curved second mold half. Arranged at the transition from the first mold half to the second mold half are a first extension and a second extension. Furthermore, the casting mold comprises a filling nozzle. This filling nozzle can be used to fill the casting mold with bone cement. The first mold half, together with the second mold half, defines a cavity intended for filling with bone cement. The first extension and the second extension together define a cutting region of the main body. At this point, the casting mold can be cut open, for example using a scalpel, to remove a spacer produced in the casting mold. The cutting region is arranged between the first extension and the second extension. The casting mold also comprises a first passage, which allows excess bone cement to escape from the cavity of the casting mold.

One embodiment of the invention thus relates to a casting mold for producing an acetabular spacer, comprising a main body with a curved first mold half and a curved second mold half, a first extension and a second extension, a filling nozzle and a first passage, wherein the first mold half together with the second mold half defines a cavity, wherein the first extension and the second extension are arranged adjacent to each other on the main body and together define a cutting region of the main body, wherein the cutting region is arranged between the first extension and the second extension, and wherein the first passage is designed to allow excess bone cement to escape from the cavity of the casting mold.

The casting mold comprises a cavity. This cavity is substantially formed by the first mold half and the second mold half. The cavity can have a smooth surface. In one embodiment, both the first mold half and the second mold half have a completely smooth inner surface. In some embodiments, the casting mold is designed to produce spacers with surface textures. For this purpose, an inner surface of the first mold half and/or an inner surface of the second mold half may exhibit corresponding textures. Examples of such textures include ribs, knobs, and depressions. Such a textured surface can, for example, facilitate the fixation of an acetabular spacer in a patient's hip joint. Therefore, it can be advantageous if the first mold half comprises an inner surface with such a texture.

The casting mold can comprise a plastics material. The casting mold can be made entirely or partially of plastics material. This allows cost-effective production, for example through an injection molding process. For example, the first mold half and the second mold half can each be produced separately from plastics material in the injection molding process and then welded or fused together. In some embodiments, the casting mold is formed in one piece. In some designs, the casting mold is free of parting lines, which can lead to flash in the spacer produced therein. In some embodiments, the first mold half is fixedly connected to the second mold half. In some embodiments, the first mold half is seamlessly connected to the second mold half. In particular, the cutting region of the casting mold can comprise a plastics material. This allows the casting mold to be easily cut open along the cutting region. In some embodiments, the plastics material is a thermoplastic. Examples of usable plastics materials include polyethylene and polypropylene. Plastics having a low density, such as LD-PE, are particularly preferred. Preferably, the plastics material has a density of less than 0.94 g/cm³ at 1 bar and 25° C.

The cutting region preferably extends circumferentially around the main body of the casting mold. In one embodiment, the cutting region extends at least along half the circumference of the main body of the casting mold. The main body of the casting mold preferably has a substantially circular cross section. Tests showed that cutting open approximately half the casting mold, i.e., cutting open the casting mold along half the circumference of the main body of the casting mold, is usually sufficient to open the casting mold substantially completely and remove the spacer produced therein.

To facilitate cutting open the casting mold along the cutting region, it can be advantageous to design the cutting region with a reduced wall thickness. In one embodiment, the main body has a first wall thickness, and the cutting region has a second wall thickness, wherein the first wall thickness is thicker than the second wall thickness. This means that the cutting region may have a reduced wall thickness compared to the rest of the main body of the casting mold.

In one embodiment, the first extension and the second extension are designed for guiding a scalpel along the cutting region to cut through the casting mold. To do this, a user of the casting mold can guide a scalpel between the first extension and the second extension along the cutting region to cut open the casting mold. The first and second extension can prevent the user from slipping while guiding the scalpel and thus injuring themselves. Like the illustration above, the first extension and the second extension can extend circumferentially around the main body of the casting mold. Preferably, the first extension and the second extension extend around at least half the circumference of the main body.

To make it easier to open the casting mold, the first extension and/or the second extension may comprise a tab. In one embodiment, the first extension comprises a tab. In one embodiment, the second extension comprises a tab. Preferably, both the first and second extension each comprise a tab. This makes it easier to manually pull apart the first and second mold half. To do this, a user of the casting mold can grasp the tabs with their hand and thereby separate the first mold half from the second mold half, pulling the two tabs apart. The spacer produced in the casting mold can then be easily removed.

In one embodiment, the first extension and/or the second extension extends at least 2mm outward from the main body. This can make it easier for a user of the device to guide a scalpel along the cutting region to cut through the casting mold. In one embodiment, the first extension extends at least 2mm outward from the main body. In one embodiment, the second extension extends at least 2mm outward from the main body. In one embodiment, both the first extension and the second extension extend at least 2mm outward from the main body. In some embodiments, the first extension and/or the second extension extends at least 3, 4, 5 or 6mm outward from the main body.

In some embodiments, the first extension and/or the second extension extends circumferentially around at least half of the circumference of the main body. This can allow a user of the device to safely guide a scalpel along the cutting region around at least half the circumference of the main body to cut open the casting mold sufficiently to allow a spacer produced therein to be easily removed from the casting mold. In one embodiment, the cutting region extends circumferentially around at least half of the circumference of the main body.

In some embodiments, the cutting region has a reduced wall thickness. In some embodiments, the cutting region has a wall thickness of at most 1mm, preferably at most 0.5mm or at most 0.4mm. This can allow a user of the casting mold to cut open the casting mold at the cutting region with minimal effort. This can help prevent injuries, as less force is required and the scalpel is less likely to slip. Furthermore, the likelihood of damaging the scalpel and/or the spacer contained in the casting mold when cutting the mold open is reduced.

In some embodiments, the casting mold comprises a means for severing a sprue. The means for severing a sprue can preferably comprise a movable element. The movable element can comprise a passage, in particular a first passage as described herein or a second passage as described herein. The movable element can comprise a projection, in particular a first projection described herein or a second projection described herein.

In some embodiments, the casting mold comprises a plug. The plug can be designed to close the filling nozzle. The plug preferably has a diameter that is smaller than the diameter of the filling nozzle. The plug can preferably be rotatably inserted into the filling nozzle. This means that the plug can still be rotated in a state where it closes off the filling nozzle, without opening the casting mold. The first passage described herein can preferably be arranged in the plug. Accordingly, the plug does not completely hermetically seal the filling nozzle, but allows excess bone cement to flow out of the casting mold when the plug is pressed into the filling nozzle.

The casting mold comprises a filling nozzle. The filling nozzle can have a substantially cylindrical shape. The filling nozzle can be closed off by a plug in the casting mold. The filling nozzle can comprise a stop to limit the penetration depth of the plug into the filling nozzle. This stop can be conveniently designed as a tapering of the filling nozzle. Accordingly, the filling nozzle can comprise a neck which is designed as a tapering of the cylindrical basic shape of the filling nozzle.

The first passage described herein preferably defines an access point through which the cavity of the casting mold can be filled with bone cement. This access point preferably forms the only position at which the spacer produced in the casting mold comprises a sprue with a width of more than 1 mm. The designs described herein can minimize this sprue or even completely prevent its formation, or the sprue can be severed by appropriate designs of the casting mold. This allows a spacer to be produced without a sprue or with only a minor sprue, where such a minor sprue is easily removable.

In one embodiment, the casting mold is designed and configured to sever a sprue using a movable element of the casting mold. The movable element can comprise a passage or a projection. The movable element can be arranged in a plug of the casting mold or in the filling nozzle of the casting mold.

In one embodiment, the casting mold is designed and configured to sever a sprue by a rotational movement of the plug relative to the filling nozzle. This function can be achieved through different designs, as described herein. For example, the plug can have a substantially cylindrical shape. The first passage can be arranged eccentrically in the plug, i.e., the first passage can be arranged offset from a central axis of the plug. In such an embodiment of the casting mold, if the plug is turned in a state in which the casting mold is filled with bone cement via the filling nozzle, the sprue can be severed as a result. This means that the bone cement in the cavity of the casting mold is no longer connected to the bone cement in the first passage.

Furthermore, the plug can comprise a first projection for severing a sprue. In one embodiment, the first projection is arranged in an interior space of the plug. The first projection is preferably arranged at the first passage. The first projection can be used to apply pressure to the bone cement in order to sever the sprue as explained above. Furthermore, the first projection can comprise a beveled cutting edge. This allows the first projection, in the manner of a knife, to more easily cut through the sprue. The cutting edge reduces the area of the first projection that can be pressed against the sprue when the plug is turned. This means that the force associated with the rotation of the plug is exerted on the sprue over a smaller area.

In this context, a “first passage” and a “first projection” preferably refer to elements that are arranged in a plug of the casting mold. In contrast, a “second passage” and a “second projection” preferably refer to elements that are arranged in the filling nozzle of the casting mold. The features and functions mentioned herein in connection with the first projection can each also be applied in a corresponding manner for the second projection explained below. All embodiments are considered in which a corresponding feature is present either only in the first projection, only in the second projection, or in both the first projection and the second projection. For example, both the first projection and the second projection can each comprise a beveled cutting edge. These two cutting edges can work together, so that the two cutting edges move against each other like scissors and together can cause the severing of a sprue.

Similarly, the casting mold can comprise a first passage and a second passage, each designed and arranged in the casting mold to be oriented linearly with respect to each other in a first configuration of the casting mold, and to be oriented offset from each other in a second configuration of the casting mold. This allows, on the one hand, the permeability of the filling nozzle for bone cement to be controlled, and on the other hand, a sprue to be severed. For this purpose, the first passage and/or the second passage can be arranged eccentrically. Furthermore, the first projection and/or the second projection can be arranged eccentrically. “Eccentric” refers, in the context of the first passage and/or the first projection, to a central axis of the plug, and, in the context of the second passage and/or the second projection, to a central axis of the filling nozzle.

In one embodiment, the casting mold comprises a second projection for severing a sprue. In some embodiments, the casting mold, as described above, can comprise a first projection and additionally a second projection. In one embodiment, the casting mold is designed and configured to sever a sprue by a rotational movement of the plug relative to the filling nozzle when the first projection is rotated relative to the second projection. For this purpose, the first projection, as described above, can preferably be arranged on the plug. The first projection can be arranged on the plug, preferably in the plug. The second projection can be arranged on the plug, preferably in the plug. The first projection can be arranged eccentrically on the plug. The second projection can be arranged eccentrically on the filling nozzle.

Furthermore, the plug and/or the filling nozzle can each comprise a plurality of passages. In one embodiment, the plug comprises two first passages. In one embodiment, the filling nozzle comprises two second passages.

In one embodiment, the plug comprises a plurality of passages which are designed and arranged point-symmetrically. In one embodiment, the filling nozzle comprises a plurality of passages which are designed and arranged point-symmetrically. In one embodiment, both the plug and the filling nozzle each comprise a plurality of passages which are designed and arranged point-symmetrically.

In one embodiment, the filling nozzle comprises a plurality of passages, wherein the total area of the plurality of passages is at least 30% of the internal cross-sectional area of the filling nozzle.

In one embodiment, the plug comprises a plurality of passages, wherein the total area of the plurality of passages is at least 30% of the internal cross-sectional area of the plug. The “internal cross-sectional area” is derived from the smallest clear width of the particular element.

In some embodiments, the plurality of passages of the plug and the plurality of passages of the filling nozzle are each designed in the same way as each other. For example, the plug can comprise two passages, each of which has the shape of a triangle in cross section, with the apex of the two triangular cross sections touching or nearly touching each other. The filling nozzle can also comprise second passages, each of which has the shape of a triangle in cross section, with the apex of the two triangular cross sections touching or nearly touching each other. By rotating the plug, the aforementioned passages can be aligned in a first configuration of the casting mold, so that bone cement can be introduced into the cavity of the casting mold through the filling nozzle. By further rotating the plug, the aforementioned passages can be offset from each other, so that no bone cement can pass through the filling nozzle. Thus, a user of the device can switch between an open state of the casting mold and a closed state of the casting mold by rotating the plug. At the same time, this can also be used to remove a sprue, as described herein. In this case, a first projection and a second projection can be turned counter to each other if necessary.

In other embodiments of the invention, such a function can also be achieved without a plug. In one embodiment, the casting mold comprises an operating element which is designed and configured to move a passage described herein and/or projection described herein in order to sever a sprue. In one embodiment, the casting mold comprises an operating element which is accessible from outside the casting mold and is designed and configured to sever a sprue. The operating element can be designed and configured to switch between an open state of the casting mold and a closed state of the casting mold, similar to the description above. The operating element can preferably be arranged on the filling nozzle. The operating element is preferably accessible from the outside, so that a user of the casting mold can operate the operating element from the outside to perform the function of the operating element. In one embodiment, the operating element is designed and configured, by connecting a dispensing device for bone cement to a casting mold according to the invention, to bring the casting mold into a first configuration in which bone cement can be introduced into the casting mold. For this purpose, a passage is arranged using the operating element as described herein so that bone cement can enter the casting mold through the filling nozzle.

In some embodiments, the filling nozzle comprises a second projection for severing a sprue. In some embodiments, the plug comprises a first projection for severing a sprue, and the filling nozzle comprises a second projection for severing a sprue. In some embodiments, the casting mold is designed and configured to sever a sprue by a rotational movement of the plug relative to the filling nozzle when the first projection is rotated relative to the second projection.

In some embodiments, the filling nozzle comprises a second passage. In some embodiments, the plug comprises a first passage and the filling nozzle comprises a second passage, wherein the casting mold is designed and configured to arrange the first passage along a common axis with the second passage in a first rotational position of the plug, and to arrange the first passage offset from such an axis of the second passage in a second rotational position of the plug.

In some embodiments, the casting mold is designed and configured to switch between an open state of the casting mold and a closed state of the casting mold by rotating a plug of the casting mold or by rotating an operating element. In some embodiments, the casting mold is designed and configured to sever a sprue by rotating a plug of the casting mold or by rotating an operating element.

The casting mold can also comprise one or more vent openings. Such a vent opening can make it possible to fill the cavity of the casting mold completely or almost completely with bone cement. This prevents air bubble inclusions in the spacer that can be produced in the casting mold. The vent opening can preferably be arranged between the first mold half and the second mold half. There can be a plurality of vent openings arranged between the first and second mold half. A plurality of vent openings can be arranged at regular intervals circumferentially around the casting mold between the first or second mold half. The vent opening can be arranged opposite the filling nozzle. There can be a plurality of vent openings arranged opposite the filling nozzle. The vent opening or vent openings preferably have a diameter of less than 1.0mm.

In this context, “bone cement” refers both to “bone cement dough,” i.e., a malleable mass that can be hardened into a solid, non-deformable material, and to the corresponding hardened bone cement.

Similarly, the term “sprue” here refers to deformable or hardened bone cement that forms a normally undesirable structure on a spacer produced in the casting mold. Such a sprue can form on a feed line to the cavity of the casting mold, particularly in the region of the first passage described herein and/or the second passage described herein.

Commercial bone cements are in many cases offered as a kit containing a liquid and a solid component. By mixing the liquid with the solid component, the user can produce a bone cement dough which can be easily molded for a limited time. A short time after mixing, the bone cement dough becomes tack free, i.e., it no longer adheres to a glove if there is light contact therewith (as defined in ISO 5833:2002). In this state, a bone cement dough can preferably be inserted into a casting mold according to the invention for producing spacers, for example using a stirring spatula, or preferably using a suitable dispensing device. Commercial bone cements are often based on polymethyl methacrylate (PMMA). The casting mold according to the invention can preferably be used with such a PMMA bone cement. However, it is also possible in principle to use other types of bone cements.

The invention further relates to the use of a casting mold described herein for the production of an acetabular spacer. For this purpose, the casting mold can be filled with bone cement as described herein, and the bone cement can be subjected to hardening in the casting mold to produce a solid acetabular spacer. The casting mold can then be opened as described herein, and the produced acetabular spacer can be removed from the casting mold.

The invention further relates to a method for producing an acetabular spacer. A casting mold described herein can be used for this purpose. The method can comprise the following steps:
providing a casting mold described herein;
filling the casting mold with bone cement;
hardening of the bone cement in the casting mold;
cutting open the main body of the casting mold in the cutting region;
separating the first mold half from the second mold half; and
removing the produced acetabular spacer from the cut-open casting mold.

The method can also include removing any sprue formed from the bone cement, as described herein.

The steps of the method described above can, in principle, be carried out in any order. In one embodiment, the method is carried out in the sequence shown above.

A further aspect of the invention is a spacer which can be produced, or is produced, using a casting mold described herein.

### EXAMPLES

The invention will be further illustrated below on the basis of examples, but these are not to be understood as limiting. It will be apparent to a person skilled in the art that other equivalent means may be used similarly in place of the features described here.

### FIGURES

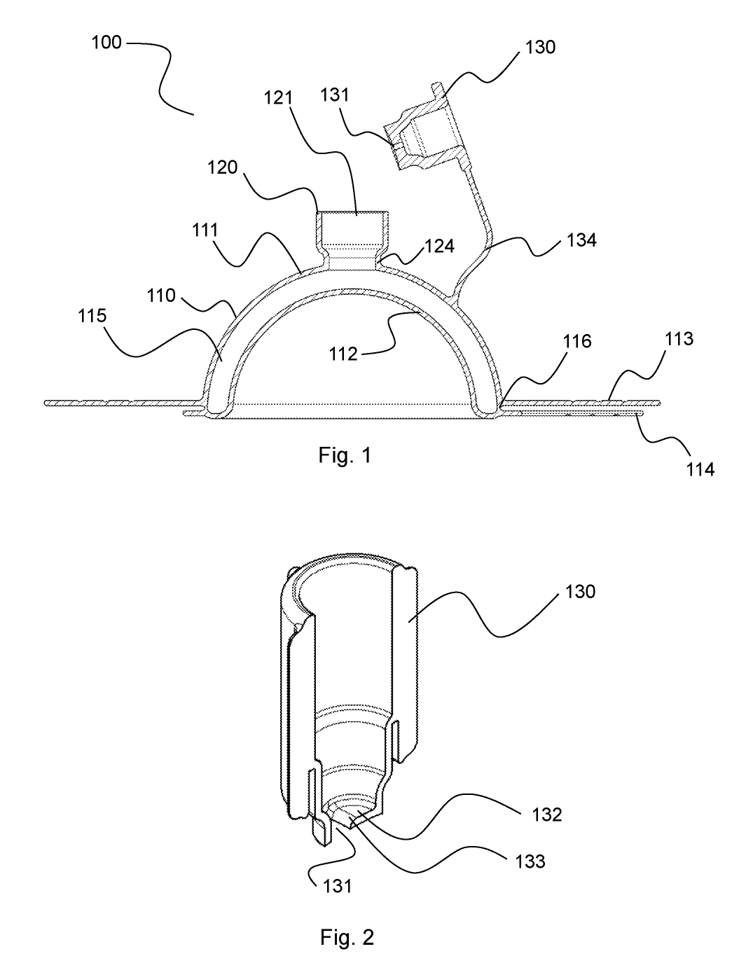

FIG. 1 shows, by way of example, a first embodiment of the invention in cross section. A casting mold 100 comprises a main body 110, which comprises a first curved mold half 111 and a second curved mold half 112. The first mold half 111 has a convexly shaped outer surface. The second mold half 112 has a concavely shaped outer surface. The first mold half 111 together with the second mold half 112 forms an inner cavity 115. This cavity 115 is designed to be filled with bone cement and corresponds to the shape of the acetabular spacer to be produced.

To fill the cavity 115, the casting mold 100 comprises a filling nozzle 120 which is arranged on the first mold half 111. The filling nozzle 120 in this case has a substantially cylindrical shape to allow a positive-locking connection to a dispensing device. The filling nozzle 120 here comprises a neck 124, which is designed as a tapering of the cylindrical basic shape of the filling nozzle 120. The neck 124 prevents the dispensing device from penetrating too deeply into the casting mold, thus preventing disruption of the molding process. The neck 124 forms a stop, so that a dispensing device cannot penetrate beyond the filling nozzle 120 into the cavity 115 between the first mold half 111 and the second mold half 112. The casting mold 100 further comprises a first extension 113 and a second extension 114, which are arranged adjacent to each other on the main body 110 and extend outward from the main body 110. The two extensions each comprise a tab so that they can each be grasped by hand by a user. The first extension 113 and the second extension 114 together define an intermediate cutting region 116 of the casting mold. This cutting region 116 is designed with a wall thickness that is smaller compared to the rest of the main body, so that the cutting region 116 can be easily cut open using a scalpel. The first extension 113 and the second extension 114 can be used here to guide the movement of the scalpel. This prevents the scalpel from slipping, thus ensuring that the casting mold 100 can be opened without causing injury. The casting mold 100 further comprises a plug 130, which is designed to close off the filling nozzle 120. The plug 130 is connected here via a link 134 to the main body 110 of the casting mold. In this way the plug 130 is continuously held on the casting mold 100 and thus it can be avoided, for example, that the plug 130 could fall down and thereby become non-sterile, for example. The plug 130 comprises a first passage 131, which is designed to produce a connection to the cavity 115. Using the first passage 131, excess bone cement can flow out of the casting mold 100 when the casting mold is closed off with the plug 130 after being filled with bone cement. The first passage 131 has a small diameter of, for example, 2mm or less, so that only a very small sprue of bone cement is created, which can be easily removed. In the embodiment shown here, the first passage 131 is concentric, i.e., is arranged along a central axis of the plug 130.

FIG. 2 shows, by way of example, an embodiment of a plug 130 in cross section. The plug 130 comprises a first passage 131 and a first projection 132. At the edge of the projection which points toward the first passage 131, the projection 132 has a beveled shape which forms a cutting edge 133. The first passage 131 is not arranged centered on the central axis of the plug 130 but instead is arranged in an eccentric position of the plug. This design of the plug 130 makes it possible to sever a sprue made of bone cement by turning the plug 130 in the filling nozzle 120. In this process, the moving cutting edge 133 of the first projection 132 cuts through the sprue. In this embodiment, it may make sense to use a plug 130 without a link 134 in order to allow free rotation of the plug 130.

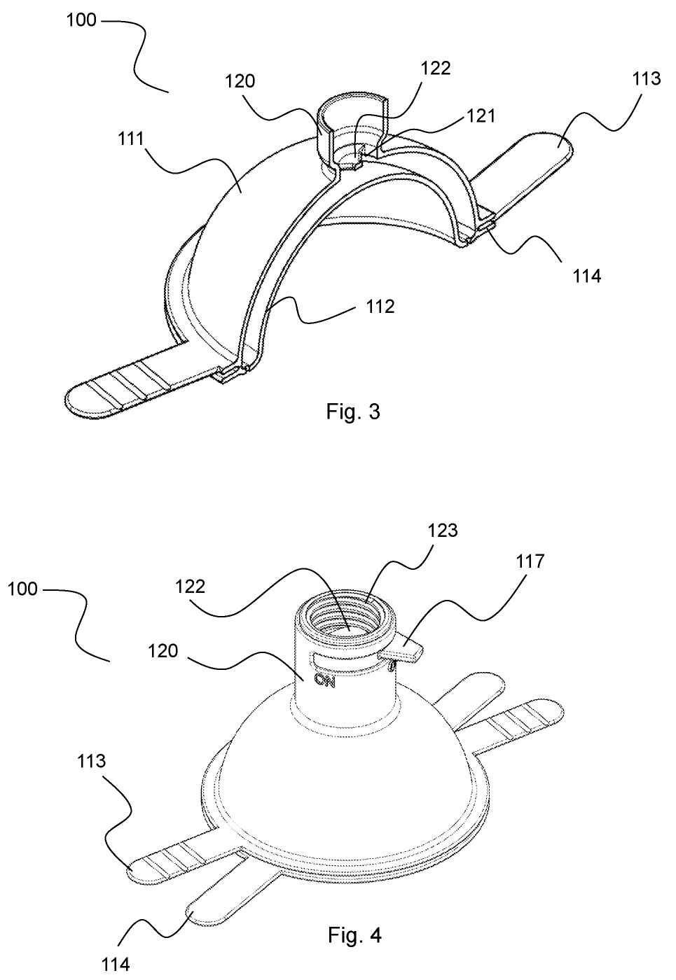

FIG. 3 shows, by way of example, a further embodiment of the invention in cross section. A casting mold 100 comprises a first mold half 111 and a second mold half 112 which are interconnected. On the first mold half 111, a filling nozzle 120 is arranged which comprises a second passage 121 and a second projection 122. The second passage 121 and the second projection 122 can be present alternatively or in addition to a first passage 131 and a first projection 132. The second passage 121 and the second projection 122 delimit the cross-sectional area of the bone cement to be received in the casting mold. This allows a thinner sprue of bone cement to be formed at the relevant location which can be easily removed.

FIG. 4 shows, by way of example, a further embodiment of the invention. The casting mold 100 shown here comprises a first extension 113 and a second extension 114, which each comprise tabs and can thereby easily be grasped with the hand by a user to separate the first mold half 111 from the second mold half 112 after the casting mold is cut open and to remove the spacer formed therein from the casting mold. The embodiment shown here comprises a filling nozzle 120 with a second projection 122 which is operatively connected to an operating element 117. The operating element 117 is accessible from the outside and allows movement of the projection 122 within the inner part of the filling nozzle 120. The movement of the projection 122 makes it possible to sever a sprue from a spacer formed in the casting mold. The filling nozzle 120 also has an internal thread 123 for connection to a bone cement dispensing device. As explained herein, the operating element 117 can be designed and configured, when connected to a dispensing device, to bring the operating element 117 into a first position, which allows the bone cement to be received into the casting mold. Furthermore, the operating element 117 can be designed and configured to sever a sprue made of bone cement by moving the operating element 117 into a second position. In some embodiments, the projection 122 in such a second position of the operating element 117 closes off the filling nozzle 120, so that no more bone cement can flow through the filling nozzle 120.

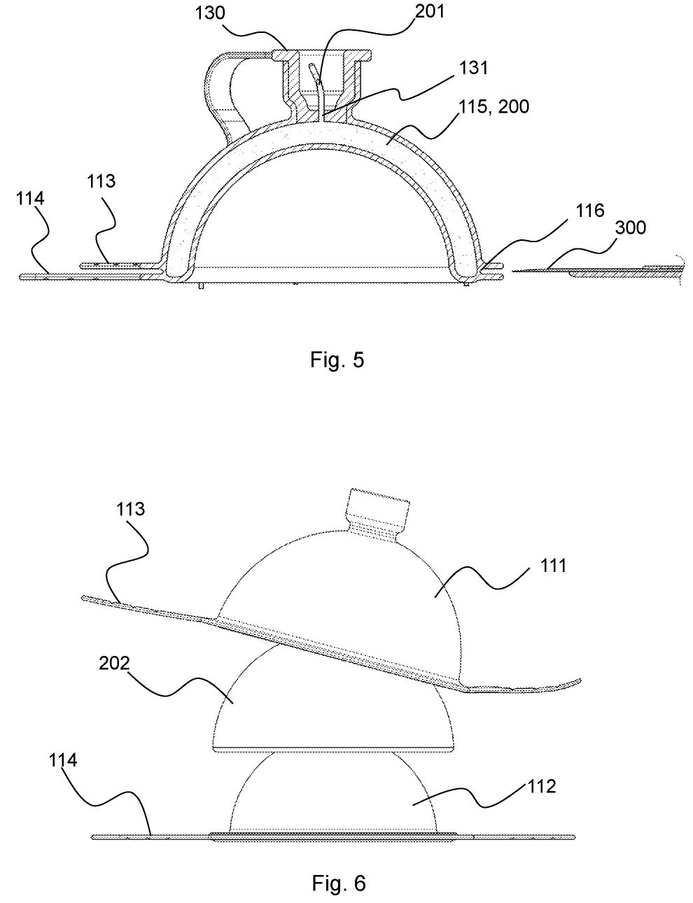

FIG. 5 shows by way of example a casting mold according to the invention which is filled with bone cement 200. The cavity 115 is completely filled with bone cement 200. By closing the casting mold with the plug 130, a slight overpressure of the received bone cement 200 is created, so that excess bone cement 200 can be released through the first passage 131 from the cavity 115 to the outside. This creates a thin sprue 201, the thickness of which is substantially determined by the diameter of the first passage 131. After the bone cement 200 has hardened, this thin sprue 201 can easily be removed from the acetabular spacer produced in the casting mold. To remove the spacer from the casting mold, it is necessary to open the casting mold. For this purpose, the casting mold can be cut open with a scalpel 300 along the circumferential cutting region 116 after the bone cement has hardened. This makes it possible to separate the first mold half 111 from the second mold half 112. Here, the first extension 113 and the second extension 114, each of which comprises a tab, can be pulled apart by hand by a user to separate the first mold half 111 from the second mold half 112.

FIG. 6 shows, by way of example, a casting mold according to the invention in which the first mold half 111 has been separated from the second mold half 112 as previously described. By grasping the first extension 113 and the second extension 114, the two mold halves can be manually pulled apart to remove the spacer 202 from the casting mold.

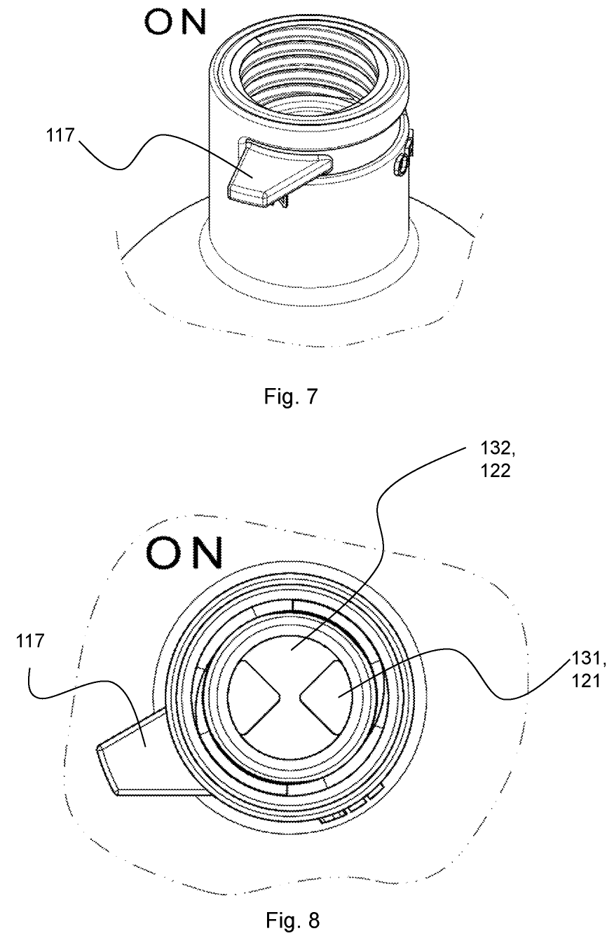

FIG. 7 shows a detail of a casting mold which, as explained in relation to FIG. 4, comprises a filling nozzle 120 with an operating element 117. The operating element 117 is located here in a first position which allows the bone cement to flow through the filling nozzle 120. This first position of the operating element 107 is marked “ON” to indicate to a user of the casting mold that, in this configuration of the casting mold, bone cement can be injected into the casting mold.

FIG. 8 shows a detail of a casting mold according to FIG. 7 with the operating element 117 being in a first position. Here, the casting mold comprises a first passage 131 and a second passage 121 which, in the first position of the operating element 117, are arranged along a common axis, thus allowing the flow of bone cement through the filling nozzle 120. Furthermore, the casting mold comprises a first projection 132 and a second projection 122 which, in the first position of the operating element 117, are aligned along a common axis.

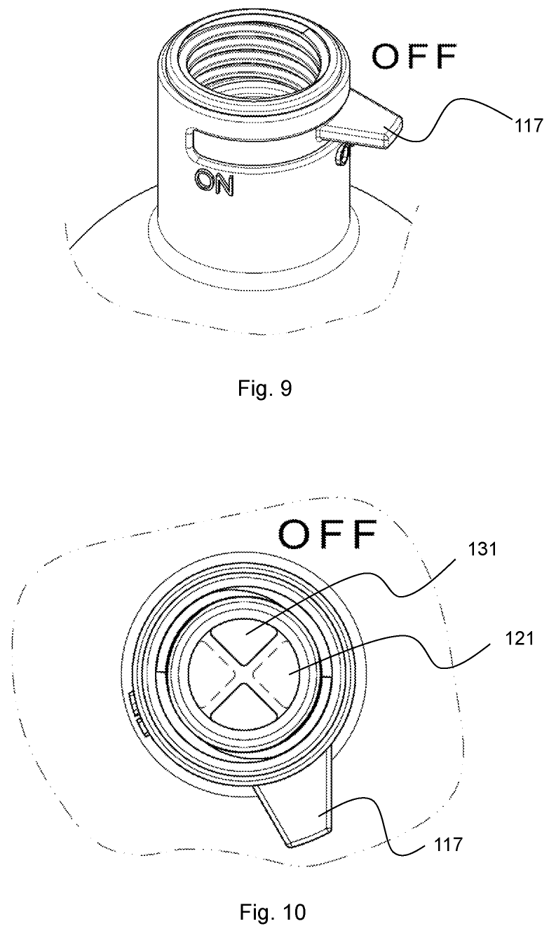

FIG. 9 shows a detail of a casting mold which, as explained in relation to FIG. 4, comprises a filling nozzle 120 with an operating element 117. The operating element 117 is located here in a second position, which prevents the bone cement from flowing through the filling nozzle 120. This second position of the operating element 107 is marked “OFF” to indicate to a user of the casting mold that in this configuration the casting mold is completely closed, so that no bone cement can flow through the filling nozzle 120.

FIG. 10 shows a detail of a casting mold according to FIG. 9, with the operating element 117 being in a second position. Here, the casting mold comprises a first passage 131 and a second passage 121 which, in the second position of the operating element 117, are arranged offset from each other, i.e., not along a common axis. The first passage 131 is thus isolated from the second passage 121. Therefore, no bone cement can pass through the filling nozzle 120. Furthermore, the casting mold comprises a first projection 132 and a second projection 122 which, also in the second position of the operating element 117, are arranged offset from each other. By moving the first projection 132 in relation to the second projection 122, the filling nozzle 120 is first blocked for the flow of bone cement and, secondly, the still malleable sprue of the bone cement is severed. This can prevent or at least significantly reduce the formation of a sprue.

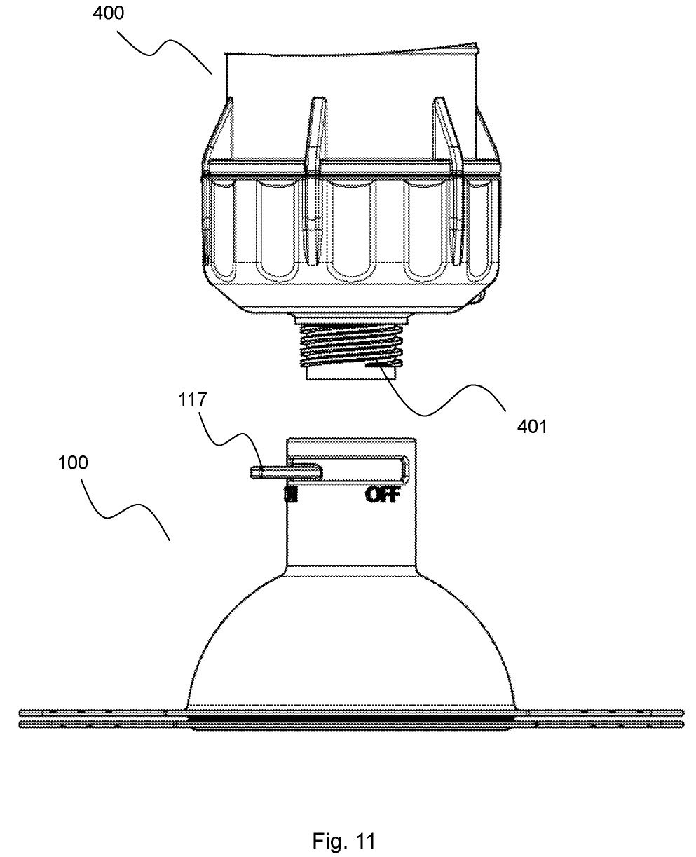

FIG. 11 shows a casting mold 100 according to the invention with an operating element 117 and a dispensing device 400 for bone cement which is arranged for connection to the casting mold 100. The dispensing device 400 has a thread 401 which can engage in a corresponding thread on the filling nozzle 120. The embodiment of the casting mold 100 shown here is designed and configured to move the operating element 117 into a first position when a dispensing device 400 is screwed into the filling nozzle 120 of the casting mold 100 with a thread 401. Thus, the casting mold 100 is automatically brought into a state that allows the bone cement from the dispensing device 400 to be received into the casting mold 100 when the dispensing device 400 is screwed into the casting mold 100.

### LIST OF REFERENCE SIGNS

100 Casting mold
110 Main body
111 First mold half
112 Second mold half
113 First extension
114 Second extension
115 Cavity
116 Cutting region
117 Operating element
118 Vent opening
120 Filling nozzle
121 Second passage
122 Second projection
123 Thread
124 Neck
130 Plug
131 First passage
132 First projection
133 Cutting edge
134 Link
200 Bone cement
201 Sprue
202 Spacer
300 Scalpel
400 Dispensing device
401 Thread

What is claimed is:

1. A casting mold for producing an acetabular spacer, comprising a main body with a curved first mold half and a curved second mold half, a first extension and a second extension, a filling nozzle and a first passage, wherein the first mold half together with the second mold half defines a cavity, wherein the first extension and the second extension are arranged adjacent to each other on the main body and together define a cutting region of the main body, wherein the cutting region is arranged between the first extension and the second extension, and wherein the first passage is designed to allow excess bone cement to escape from the cavity of the casting mold.

2. The casting mold according to claim 1, wherein the main body has a first wall thickness which is thicker than a second wall thickness of the cutting region.

3. The casting mold according to claim 1, wherein the first extension and the second extension are designed for guiding a scalpel along the cutting region to cut through the casting mold.

4. The casting mold according to claim 1, wherein the first extension and/or the second extension comprises a tab to facilitate manually pulling apart the first mold half and the second mold half.

5. The casting mold according to claim 1, wherein the first extension and/or the second extension extends at least 2 mm, more preferably at least 3, 4, 5 or 6 mm, outward from the main body.

6. The casting mold according to claim 1, wherein the first extension and/or the second extension extends circumferentially around at least half of a circumference of the main body.

7. The casting mold according to claim 1, further comprising a means for severing a sprue, wherein the means preferably comprises a movable element, wherein the movable element is further preferably selected from a passage and a projection.

8. The casting mold according to claim 1, further comprising a plug for closing the filling nozzle, wherein the first passage is preferably arranged in the plug.

9. The casting mold according to claim 8, wherein the casting mold is designed and configured to sever a sprue by a rotational movement of the plug relative to the filling nozzle.

10. The casting mold according to claim 8, wherein the plug comprises a first projection for severing a sprue, wherein the first projection is preferably arranged at the first passage.

11. The casting mold according to claim 10, wherein the first projection comprises a beveled cutting edge.

12. The casting mold according to claim 8, wherein the filling nozzle comprises a second projection for severing a sprue, wherein the casting mold preferably further comprises a first projection, wherein the plug comprises a first projection for severing a sprue, wherein the first projection is preferably arranged at the first passage and wherein the casting mold is preferably designed and configured to sever a sprue by a rotational movement of the plug relative to the filling nozzle when the first projection is rotated relative to the second projection.

13. The casting mold according to claim 8, wherein the first passage is arranged eccentrically in the plug.

14. The casting mold according to claim 8, wherein the filling nozzle further comprises a second passage, wherein the casting mold is designed and configured to arrange the first passage along a common axis with the second passage in a first rotational position of the plug, and to arrange the first passage outside a common axis with the second passage in a second rotational position of the plug.

15. The casting mold according to claim 1, further comprising an operating element which is accessible from outside the casting mold and is designed and configured to sever a sprue.

16. The casting mold according to claim 1, further comprising a vent opening, wherein the vent opening is preferably arranged between the first mold half and the second mold half.

17. A use of a casting mold according to claim 1 for producing an acetabular spacer.

18. A method for producing an acetabular spacer, comprising the steps of:
providing a casting mold according to claim 1;
filling the casting mold with bone cement;
hardening of the bone cement in the casting mold;
removing any sprue formed from the bone cement;
cutting open the main body of the casting mold in the cutting region;
separating the first mold half from the second mold half ; and,
removing the produced acetabular spacer from the cut-open casting mold.

19. The casting mold according to claim 2, wherein the first extension and the second extension are designed for guiding a scalpel along the cutting region to cut through the casting mold.

20. The casting mold according to claim 9, wherein the plug comprises a first projection for severing a sprue, wherein the first projection is preferably arranged at the first passage.
